(19) United States
(12) Patent Application Publication
(10) Pub. No.: US 2007/0073131 A1
(43) Pub. Date: Mar. 29, 2007

(54) Garment for measuring physiological signal

(76) Inventors: Chang Yong Ryu, Daejeon-city (KR); Seung Hwan Kim, Daejeon-city (KR); Seung Hoon Nam, Daejeon-city (KR); Seung Chul Shin, Daejeon-city (KR); Hyun Young Lee, Daejeon-city (KR); Kyung Hi Hong, Daejeon-city (KR)

Correspondence Address: LADAS & PARRY LLP, 224 SOUTH MICHIGAN AVENUE, SUITE 1600, CHICAGO, IL, 60604, US

(21) Appl. No.: 11/523,989

(22) Filed: Sep. 20, 2006

(30) Foreign Application Priority Data
Sep. 23, 2005 (KR) 10-2005-0088699

(51) Int. Cl. A61B 5/04

(52) U.S. Cl. 600/388; 600/389

(57) ABSTRACT

Provided is a garment for measuring a physiological signal. The garment includes: an inner layer made of an elastic mesh fabric; a physiological signal measuring electrode including a connector connected to an external line and an electrode having a protrusion and measuring a physiological signal, the protrusion of the electrode passing through a mesh of the inner layer and engaging with the connector to be attached to the inner layer; and a compressing cushion interposed between an outer layer and the inner layer and compressing the physiological signal measuring electrode to a human body. Accordingly, poor contact between the physiological signal measuring electrode and the human body can be prevented and noise generated when measuring the physiological signal can be reduced.

### CROSS-REFERENCE TO RELATED PATENT APPLICATION

This application claims the benefit of Korean Patent Application No. 10-2005-0088699, filed on Sep. 23, 2005, in the Korean Intellectual Property Office, the disclosure of which is incorporated herein in its entirety by reference.

### BACKGROUND OF THE INVENTION

1. Field of the Invention

The present invention relates to a garment for measuring a physiological signal, and more particularly, to a garment for measuring a physiological signal, which enables a physiological signal measuring electrode attached to an inner layer of the garment to be maintained in close contact with a human body.

2. Description of the Related Art

With the recent development of information and communication technologies, the demand for health monitoring systems that can measure physiological signals in daily life has increased. Particularly, this demand has increased as more elderly live alone in line with the national trend toward an aging society. Accordingly, there have been suggested various methods of measuring a physiological signal in daily life when many movements occur.

In one approach used to monitor a physiological signal, sensors for measuring a variety of physiological signals are attached to a garment and measured physiological signals are transmitted to a computer by wire or wireless means. In this approach, it is essential to minimize noise due to the movement of a user.

A method of fixing an electrode using a highly elastic and flexible band is disclosed in PCT International Patent No. WO03/095020, entitled “Garment and Method for Producing the Same”. A method of fixing an electrode by folding a textile garment at a dividing line disposed half way such that a second part forms an outer layer and a first part forms an inner layer is disclosed in PCT International Patent No. WO 03/094717, entitled “Textile Article Having Electrically Conductive Portions and Method for Producing the Same”. However, those methods have a disadvantage in that the garment moves when a user moves during daily work, thereby causing a poor contact between the electrode and a human body.

### SUMMARY OF THE INVENTION

The present invention provides a garment for measuring a physiological signal, which enables a physiological signal measuring electrode attached to an inner layer of the garment to be maintained in close contact with a human body even during a severe movement, and reduces noises generated when measuring the physiological signal.

According to an aspect of the present invention, there is provided a garment for measuring a physiological signal, comprising: an inner layer made of an elastic mesh fabric; a physiological signal measuring electrode including a connector connected to an external line and an electrode having a protrusion and measuring a physiological signal, the protrusion of the electrode passing through a mesh of the inner layer and engaging with the connector to be attached to the inner layer; and a compressing cushion interposed between an outer layer and the inner layer and compressing the physiological signal measuring electrode to a human body.

According to another embodiment of the present invention, there is provided a garment for measuring a physiological signal, comprising: an outer layer; an inner layer; at least one physiological signal measuring electrode attached to the inner layer and measuring a physiological signal; a support vertically attached to the inner layer and supporting the garment; and a module pocket disposed at a side of the inner layer and containing a physiological signal measuring module, wherein a fastening member of the outer layer and a fastening member of the inner layer are disposed at different positions of the human body when the outer layer and the inner layer are worn on the human body.

Accordingly, poor contact between the physiological signal measuring electrode and the human body can be prevented and noise generated when measuring the physiological signal can be reduced.

### BRIEF DESCRIPTION OF THE DRAWINGS

The above and other features and advantages of the present invention will become more apparent by describing in detail exemplary embodiments thereof with reference to the attached drawings in which:

FIGS. 1, 2A, and 2B illustrate a physiological signal measuring garment to which an electrode signal measuring electrode is attached, according to an embodiment of the present invention;

FIGS. 3A and 3B illustrate an inner layer and an outer layer of the physiological signal measuring garment of FIG. 1, according to an embodiment of the present invention;

FIGS. 4A and 4B illustrate an inner layer and an outer layer of the physiological signal measuring garment of FIG. 1, according to another embodiment of the present invention;

FIG. 5 illustrates an example where the physiological signal measuring garment of FIG. 1 is worn by a user; and

FIG. 6 is a block diagram of a physiological signal measuring module according to an embodiment of the present invention.

### DETAILED DESCRIPTION OF THE INVENTION

The present invention will now be described more fully with reference to the accompanying drawings, in which preferred embodiments of the invention are shown.

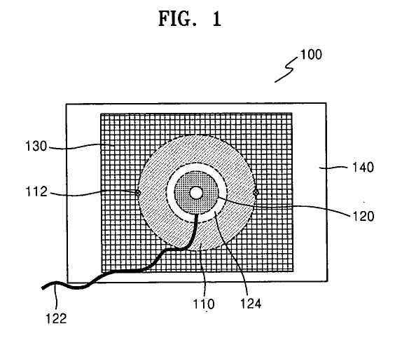

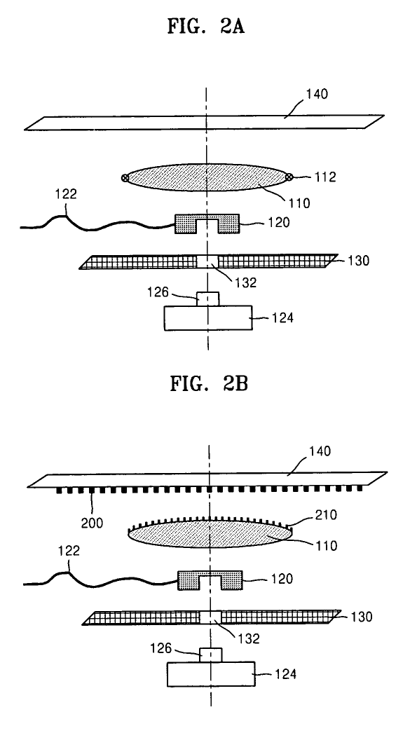

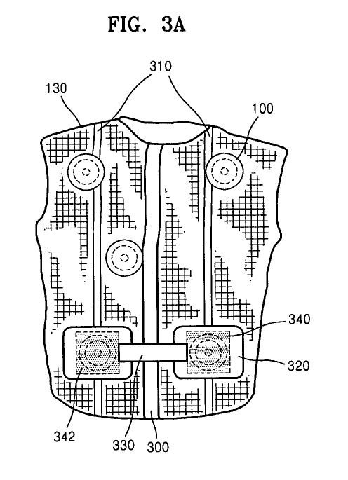

FIGS. 1, 2A, and 2B illustrate a physiological signal measuring garment to which a physiological signal measuring electrode is attached, according to an embodiment of the present invention.

Referring to FIGS. 1 through 2B, a physiological signal measuring garment 100 includes an outer layer 140, an inner layer 130, a physiological signal measuring electrode having an electrode connector 120 and an electrode 124 and passing through the inner layer 130 to be attached to the inner layer 130, and a compressing cushion 110 interposed between the outer layer 140 and the inner layer 130 and compressing the physiological signal measuring electrodes 120 and 124 to a user's body.

The inner layer 130 is made of an elastic mesh fabric through which a protrusion 126 of the electrode 124 can pass. Accordingly, a contact area between the inner layer 130 and the skin of the human body is reduced to increase air permeability. As a result, the amount of sweat generated when the garment 100 composed of the two layers 130 and 140 is worn can be reduced, thereby improving comfortability. Also, the mesh inner layer 130 can be more easily extended, contracted, and sheared than a general textile or a knit, thereby easily responding to the extension or contraction of the skin of the human body.

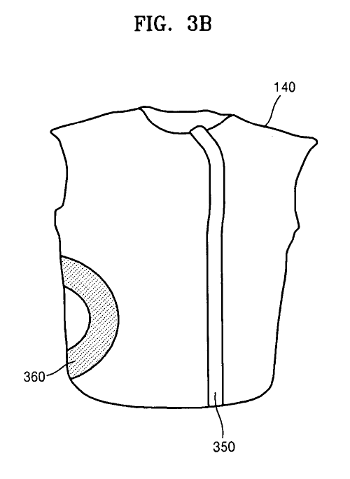

Each of the protrusion 126 formed at a central portion of the electrode 124 and the electrode connector 120 engage with the inner layer 130 therebetween. The electrode connector 120 is connected to a physiological signal measuring module 340 or 342 (see FIG. 3A) via a cable 122. The electrode 124 is in direct contact with the skin, and may be a disposable electrode. The electrode 124 may be a dryness electrode or a conductive rubber electrode having conductivity and adhesiveness. When the electrode 124 is a conductive rubber electrode, a surface of the electrode 124 contacting the skin may be concave. Then, when a slight pressure is applied to the concave surface, the concave surface may be stuck to the skin.

Referring to FIG. 2A, the compressing cushion 110 is interposed between the outer layer 140 and the inner layer 130 to correspond to the physiological signal measuring electrode and is attached to an outer surface of the inner layer 130 using needle knots 112. The compressing cushion 110 tightly compresses the physiological signal measuring electrode, which includes the electrode connector 120 and the electrode 124 and is attached to the inner layer 130, to the skin due to the high elasticity of the outer layer 140. The needle knots 112 are made at intervals of 180 degrees when the electrode connector 120 and the elect to minimize a possibility that the cable 122 is caught by the knots rode 124 of the physiological signal measuring electrode are attached to the inner layer 130.

Referring to FIG. 2B, the compressing cushion 110 is conveniently attached to the outer layer 140, without needle knots, by attaching a smooth roof portion 200 of a Velcro to an inner surface of the outer layer 140 and attaching a rough hook portion 210 of the Velcro to an upper surface of the compressing cushion 110 which contacts the inner surface of the outer layer 140. In this case, the compressing cushion 110 can be attached to the outer layer 140 considering a varied position of the physiological signal measuring electrode with the electrode connector 120 and the electrode 124.

FIGS. 3A and 3B illustrate the inner layer 130 and the outer layer 140 of the physiological signal measuring garment of FIG. 1, respectively, according to an embodiment of the present invention.

Referring to FIGS. 3A and 3B, when fastening members 300 and 350, such as zippers, of the inner layer 130 and the outer layer 140 are disposed at the same position in the middle of the human body, if the user bends downward, the fastening members 300 and 350 having no elasticity are folded and are projected forward, thereby making poor contact between the physiological signal measuring electrode and the skin. To solve this problem, the fastening member 300 of the inner layer 130 is disposed in the middle of the human body, and the fastening member 350 of the outer layer 140 is disposed on the left or right side of the human body, such that the fastening member 300 of the inner layer 130 does not project forward due to the elasticity of the outer layer 140, thereby preventing poor contact between the physiological signal measuring electrode and the skin.

Princess lines 310, that is, supports, extending from a front surface to a rear surface of the inner layer, support the mesh inner layer 130 to avoid the distortion of the garment. The princess lines 310 hold the weights of pockets 320 containing the flexible physiological signal measuring modules 340 and 342. One or more loops or Velcros may be attached to each of the princess lines 310 to fix the cable 122 in the vicinity of a physiological signal measuring electrode 100. A sensor fixing rubber band attached to the princess line 310 is small enough to fix sensor connecting lines in ordinary times, and has elasticity high enough to allow a head of a sensor to be easily pass therethrough when a sensor is attached or detached.

The physiological signal measuring module pockets 320 have an waterproof inner surface to prevent entrance of external moisture, and is made of a soft cloth such that the physiological signal measuring modules 340 and 342 can smoothly conform with the curved shape of the human body.

When the two physiological signal measuring modules 340 and 342 are attached to an abdomen portion of the garment, the weight is balanced on the left and right sides. A cable 330 connecting the two physiological signal measuring modules 340 and 342 may be used as a power switch. For example, when the two physiological signal measuring modules 340 and 342 are connected, the switch is turned on, and when they are separated from each other, the switch is turned off. The two physiological signal measuring modules 340 and 342 may be disposed at a chest portion of a physiological signal measuring garment for men. As shown in FIG. 3B, a cloth 360 with low elasticity may be additionally attached to the outside of the outer layer 140, corresponding to the physiological signal measuring electrode, to increase the degree of contact between the physiological signal measuring electrode and the skin.

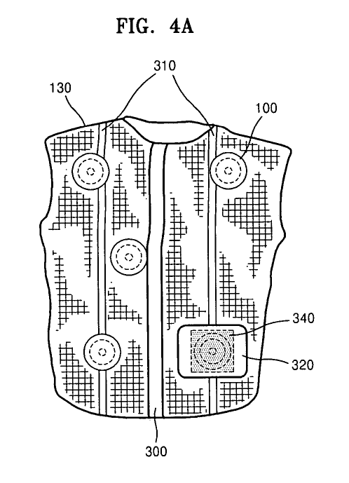

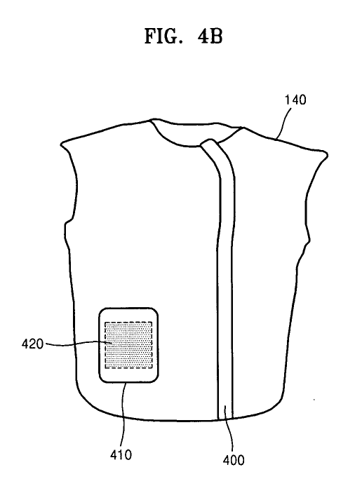

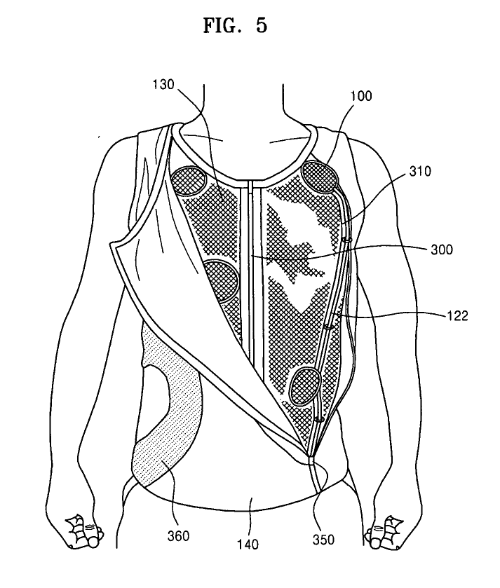

FIGS. 4A and 4B illustrate an inner layer and an outer layer of the physiological signal measuring garment of FIG. 1 according to another embodiment of the present invention.

Referring to FIGS. 4A and 4B, a pocket 410 is disposed outside the outer layer 140 such that one of two physiological signal measuring modules of the inner layer 130 is disposed outside. To balance the weight of the physiological signal measuring garment, the two physiological signal measuring module pockets 320 and 410 are disposed at left and right sides, respectively. A transparent window 420 is formed at a central portion of the outer pocket 410, such that the state of a physiological signal collected and transmitted by the physiological signal measuring module 342 can be seen through the transparent window 420.

FIG. 5 illustrates an example where the physiological signal measuring garment of FIG. 1 is worn by a user.

Referring to FIG. 5, the physiological signal measuring electrode 100 is attached to the inner surface of the mesh inner layer 130, the physiological signal measuring module is contained in the pocket attached to the outer surface of the inner layer 130, and the outer layer 140 is worn on the body. The fastening member 300 of the inner layer 130 is disposed in the middle of the human body, and the fastening member 350 of the outer layer 140 is disposed on the left or right side of the human body, such that the two fastening members 300 and 350 do not overlap each other. The cloth 360 with low elasticity is additionally attached to the outside of the outer layer 140 to correspond to the physiological signal measuring electrode 100 such that the physiological signal measuring electrode 100 can be in close contact with the skin.

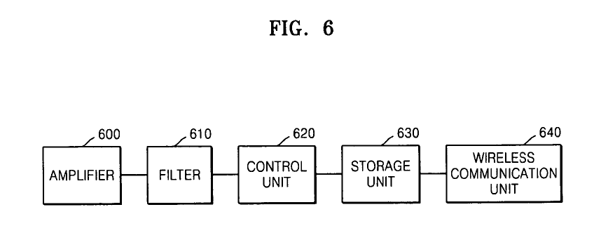

FIG. 6 is a block diagram of a physiological signal measuring module according to an embodiment of the present invention.

Referring to FIG. 6, each of the physiological signal measuring modules 340 and 342 (see FIG. 3) includes an amplifier 600, a filter 610, a control unit 620, a storage unit 630, and a wireless communication unit 640. The physiological signal measuring modules 340 and 342 contained in the pockets 320 of the inner layer 130, or the pocket 320 of the inner layer 130 and the pocket 410 of the outer layer 140, analyze a physiological signal measured by the physiological signal measuring electrode 100, and display the analysis result to the user.

In detail, the amplifier 600 amplifies the physiological signal measured by the physiological signal measuring electrode 100. The filter 610 removes noise from the amplified physiological signal. The control unit 620 converts the noise-free analogue physiological signal into a digital signal. The storage unit 630 stores the physiological signal. The wireless communication unit 640 transmits the physiological signal digitalized by the control unit 620 to the outside via a wired or wireless communication network, such as the Internet or Bluetooth.

As described above, since the mesh inner layer 130 can be more easily extended, contracted, and sheared than a general textile or a knit, the inner layer 130 can easily respond to the extension or contraction of the skin. Since the high elastic outer layer 140 placed over the inner layer 130 tightly attaches the physiological signal measuring electrode 100 and the compressing cushion 112, which is attached between the outer surface of the inner layer 130 and the inner surface of the outer layer 140, to the skin at the same time and the low elastic cloth 360 is additionally applied to the high elastic outer layer 140 to correspond to the physiological signal measuring electrode 100, the degree of contact between the physiological signal measuring electrode 100 and the skin can be stably maintained despite the curved shape or the movement of the human body. Accordingly, a contact resistance between the skin and the physiological signal measuring electrode 100 can be reduced, and noise generated when measuring the physiological signal can be reduced as well.

When the Velcro is applied to the compressing cushion 112, since the position of the electrode 100 over the body can be changed freely, a physiological signal can be measured at various positions.

Since the fastening member 300 of the inner layer 130 and the fastening member 350 of the outer layer 140 are disposed at different positions, the fastening member 300 of the inner layer 300 is prevented from projecting forward according to the movement of the human body and from causing poor contact between the electrode 100 and the skin.

Since a physiological signal can be monitored while stably maintaining the contact between the electrode 100 and the skin during daily work activities by means of the physiological signal measuring modules 340 and 342 that are coupled to the garment to transmit physiological signal data in a wireless manner, the health state of the user can be monitored for a long time, thereby allowing a user to take proper and rapid measures in case of illness. Also, the locomotion of a user can be improved by analyzing a variation in the physiological signal data.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A garment for measuring a physiological signal, comprising:
an inner layer made of an elastic mesh fabric;
a physiological signal measuring electrode including a connector connected to an external line and an electrode having a protrusion and measuring a physiological signal, the protrusion of the electrode passing through a mesh of the inner layer and engaging with the connector to be attached to the inner layer; and
a compressing cushion interposed between an outer layer and the inner layer and compressing the physiological signal measuring electrode to a human body.

2. The garment of claim 1, wherein the physiological signal measuring electrode is a dryness electrode or a rubber electrode having conductivity and adhesiveness.

3. The garment of claim 1, wherein a surface of the physiological signal measuring electrode contacting the human body is concave.

4. The garment of claim 1, wherein the compressing cushion is fixed to the inner layer using a thread.

5. The garment of claim 1, wherein the compressing cushion is attached to the outer layer using a Velcro.

6. A garment for measuring a physiological signal, comprising:
an outer layer;
an inner layer;
at least one physiological signal measuring electrode attached to the inner layer and measuring a physiological signal;
a support vertically attached to the inner layer and supporting the garment; and
a module pocket disposed at a side of the inner layer and containing a physiological signal measuring module,
wherein a fastening member of the outer layer and a fastening member of the inner layer are disposed at different positions of the human body when the outer layer and the inner layer are worn on the human body.

7. The garment of claim 6, wherein the fastening member of the inner layer is disposed in the middle of the human body and the fastening member of the outer layer is disposed on the left or right side of the human body.

8. The garment of claim 6, wherein the inner layer is made of an elastic mesh fabric; and
the physiological signal measuring electrode includes a connector connected to an external line and an electrode having a protrusion and measuring a physiological signal, the protrusion of the electrode passing through a mesh of the inner layer and engaging with the connector to be attached to the inner layer,
the garment further comprising a compressing cushion interposed between the outer layer and the inner layer and compressing the physiological signal measuring electrode to a human body.

9. The garment of claim 6, wherein the support includes loops disposed at predetermined intervals to fix a cable that connects the physiological signal measuring electrode to the physiological signal measuring module contained in the module pocket.

10. The garment of claim 6, further comprising a connecting unit connecting physiological signal measuring modules contained in module pockets disposed on left and right sides of the inner layer,
wherein, when the connecting unit connects the physiological signal measuring modules, the physiological signal measuring modules are turned on.

11. The garment of claim 6, further comprising an outer pocket disposed on the outer layer and containing a physiological signal measuring module, the outer layer having a transparent window through which the state of a physiological signal collected and transmitted by the physiological signal measuring module can be seen.

12. The garment of claim 6, wherein a cloth having low elasticity is additionally applied to a portion of the outer layer corresponding to the physiological signal measuring electrode to increase the degree of contact between the physiological signal measuring electrode and the human body.

13. The garment of claim 6, wherein the physiological signal measuring module amplifies a physiological signal received from the physiological signal measuring electrode, removes noise from the amplified signal, and transmits the noise-free signal to the outside.

14. The garment of claim 13, wherein the physiological signal measuring module comprises:
an amplifier amplifying a physiological signal measured by the physiological signal measuring electrode;
a filter removing noise from the amplified physiological signal; and
a wireless communication unit transmitting to the noise-free physiological signal via a wireless communication network such as the Internet and Bluetooth.
